SEQUENCE LISTING (2 sequences)

SEQ ID NO: 1 - DNA, 20 bp, Artificial Sequence
  Forward qPCR primer
tgacatccacggaaccctcc

SEQ ID NO: 2 - DNA, 19 bp, Artificial Sequence
  Reverse qPCR primer
gacgctgcctccattgctg

(19) United States
(12) Patent Application Publication
(10) Pub. No.: US 2022/0177902 A1
(43) Pub. Date: Jun. 9, 2022

(54) LOW-METHANE RICE

(71) Applicant: Chuanxin SUN, Uppsala (SE)

(72) Inventors: Chuanxin SUN, Uppsala (SE); Anna SCHNÜRER, Uppsala (SE); Yunkai JIN, Uppsala (SE); Ali MOAZZAMI, Uppsala (SE); Mathilde BETTEMBOURG, Uppsala (SE); Jia HU, Uppsala (SE)

(21) Appl. No.: 17/442,179

(22) PCT Filed: Mar. 23, 2020
(86) PCT No.: PCT/SE2020/050304

§ 371 Date: Sep. 23, 2021

Related U.S. Application Data
(60) Provisional application No. 62/825,626, filed on Mar. 28, 2019.

(51) Int. Cl. C12N 15/82

(52) U.S. Cl. CPC C12N 15/8243

(57) ABSTRACT

The present invention relates to a rice plant material capable of reducing methane emission by reducing organic acid, such as fumarate, secretion from the roots of the rice plant. The reduction in organic acid, such as fumarate, secretion reduces the amount of methanogens associated with the roots of the rice plant and thereby reduces the emission of methane from such methanogens.

### TECHNICAL FIELD

The present invention generally relates to low-methane rice and in particular to rice plant material capable of reducing methane emissions from rice paddies.

### BACKGROUND

Rice is a main staple food in the world and over half of the human population eats rice as a staple food. Yearly production of rice is around 700 million tons. Rice agriculture is the largest anthropogenic source of atmospheric methane. The scenario is exacerbated by the expanding rice cultivation to meet the escalating demand for food in the coming decades. In fact, atmospheric methane has made a 20% contribution to global warming since preindustrial times.

Therefore, there is an urgent need to establish sustainable technologies that allow for intensification of rice production while reducing methane fluxes from paddy fields. Strategies for developing high-yielding rice as a mean to curb methane emissions were proposed in 2002. However, as of yet no such “high-yield-low-methane” rice has been reported.

### SUMMARY

It is a general objective to obtain a rice plant material capable of reducing methane emissions from rice paddies.

This and other objectives are met by embodiments as disclosed herein.

The present invention relates to a rice plant material capable of reducing methane emission by reducing organic acids secretion, such as fumarate secretion and/or malate secretion, from the roots of the rice plant. This reduction in organic acids secretion reduces the amount of methanogens associated with the roots of the rice plant and thereby reduces the emission of methane from such methanogens.

The present invention is defined in the independent claims. Further embodiments of the invention are defined in the dependent claims.

An aspect of the invention relates to a method of producing a low-methane rice plant. The method comprises modifying a rice plant material for reduced organic acid secretion from roots of the rice plant material or from roots of a rice plant obtained from the rice plant material. In this embodiment, an amount of organic acids secreted from the roots of the rice plant material or of the rice plant is equal to or less than 90% of an amount of the organic acids secreted from roots of a corresponding wild-type rice plant lacking the modification. The reduced organic acid secretion from the roots of the rice plant material or of the rice plant induces a reduction in methane emission from methanogens present in connection with the roots of the rice plant material or of the rice plant.

Another aspect of the invention relates to a method of reducing emission of methane from a rice paddy. The method comprises deleting a sucrose-responsive region in the SUSIBA1 promoter in a rice plant material. The method also comprises cultivating the rice plant material or a rice plant obtained from the rice plant material in a rice paddy.

A further aspect of the invention relates to a method of reducing emission of methane from a rice paddy. The method comprises reallocating carbon from roots of a rice plant into panicles of the rice plant. The method also comprises cultivating the rice plant in a rice paddy, wherein carbon reallocation causes a reduction in production and secretion of organic acids from the roots and thereby reduced methane emission from methanogens present in the soil in connection with the roots in the rice paddy.

The present invention can be used to reduce methane emissions from rice paddies, thereby contributing to the usage of rice as food but with less negative impact on global warming due to increase in atmospheric methane.

### BRIEF DESCRIPTION OF THE DRAWINGS

The embodiments, together with further objects and advantages thereof, may best be understood by making reference to the following description taken together with the accompanying drawings, in which:

FIG. 1. Schematic representation of rice growth conditions. Temporal sampling is at Week 4, Week 6, Week 8, Week 13 and Ripening and spatial sampling at different temporal time is indicated as Horizontal Positions 1-3 (H. Position 1-3) and Vertical Position 1-3 (V. Position 1-3).

FIG. 2. Phenotyping of rice roots at Week 4, 6 and 7. Photographs of rice roots (FIG. 2A) and root length (FIG. 2B). *P<0.05 is shown for significant differences between SUSIBA2 rice and Nipponbare. Bars=2 cm.

FIG. 3. Methane emissions from SUSIBA2 rice and Nipponbare at different time courses. *P<0.05 and **P<0.01 are shown for significant differences between SUSIBA2 rice and Nipponbare.

FIG. 4. Fractionation of rice roots and rhizosphere regions (FIG. 4A), and microbe determination of Total fractions at Week 13 for horizontal positions (FIG. 4B). Mst (Methanosaetaceae), MET (methanogens), Msc (Methanosarcinaceae), MBT (Methanobacteriales), MMB (Methanomicrobiales), Arc (archaea), and Met for Methanocella-specific. *P<0.05 and **P<0.01 are shown for significant reduction of microbe levels of SUSIBA2 rice compared with Nipponbare.

FIG. 5. Microbe determination of the fractions soil (FIG. 5A) and rhizosphere (FIG. 5B) at Week 13 for horizontal positions. *P<0.05 and **P<0.01 are shown for significant reduction of microbe levels of SUSIBA2 rice compared with Nipponbare.

FIG. 6. Microbe determination of the fractions rhizoplane (FIG. 6A) and endosphere (FIG. 6B) at Week 13 for horizontal positions. *P<0.05 and **P<0.01 are shown for significant reduction of microbe levels of SUSIBA2 rice compared with Nipponbare.

FIG. 7. Microbe determination of total fraction at Week 4 (FIG. 7A) and Week 6 (FIG. 7B) for vertical positions. *P<0.05 and **P<0.01 are shown for significant reduction of microbe levels of SUSIBA2 rice compared with Nipponbare.

FIG. 8. NMR analysis of root exudates at Week 4, Week 6, Week 8 and Ripening.

FIG. 9. RNAseq analysis of SUSIBA2 rice roots compared with Nipponbare at Week 6. Schematic representation of 600 up- and 889 down-regulated genes in SUSIBA2 rice roots (FIG. 9A). A down-regulated gene in the Krebs cycle with SUSIBA2 rice roots (FIG. 9B).

FIG. 10. Relative abundance of bacterial and archaeal phyla (FIG. 10A). Relative abundance of archaea class in samples taken from soil A) Fuijan; soil B) Nanning, root P) phytotron and soil P) phytotron during cultivation of the rice varieties Nipponbare (Nipp), SUSIBA2-77, and SUSIBA2-80 (Su et al, 2015) (FIG. 10B). Each sample is analyzed in triplicates.

FIG. 11. CRISPR/Cas deletion of the barley corresponding sugar-sensing sequence in the rice SUSIBA1 promoter leads to an increase in panicle length. Corresponding sugar-sensing sequence and schematic diagram of CRISPR/Cas deletion in the rice SUSIBA1 promoter (FIG. 11A). Nipp: The wild type rice variety Nipponbare, which has been exposed to the same protocol of CRISPR tissue culture without deletion. CRISPR: Nipponbare rice with CRISPR/Cas deletion of the SUSIBA1 sequence (FIG. 11B). ** indicates that the statistical difference between long and short panicles is significant (P<0.01). n=12. CRISPR rice and Nipponbare rice were cultivated under the same phytotron conditions as described (Su et al. (2015)).

FIG. 12. Response of methanogen propagation of different groups upon fumarate treatment for two weeks (from Week 6 to Week 8). Mst (Methanosaetaceae), MET (methanogens), Msc (Methanosarcinaceae), MBT (Methanobacteriales), MMB (Methanomicrobiales), Arc (archaea), and Met for Methanocella-specific.

FIG. 13. qPCR analysis of genes for three rate limiting enzymes (citrate synthase (E.C. 2.3.3.1), isocitrate dehydrogenase (EC 1.1.1.42 and EC 1.1.1.41) and α-ketoglutarate dehydrogenase (EC 1.2.4.2, EC 2.3.1.61 and EC 1.8.1.4) and key enzymes for fumarate accumulation (succinic dehydrogenase EC 1.3.5.1, fumarase EC 4.2.1.2 and malate dehydrogenase EC 1.1.1.37). RNA from Week 6 was used for gene expression analysis. *P<0.05 and **P<0.01 are shown for significant differences between SUSIBA2 rice and Nipponbare.

FIG. 14. Copy number of Geobacter sulfurreducens in rhizosphere of rice roots (copies g-DW-root⁻¹).

### DETAILED DESCRIPTION

The present invention generally relates to low-methane rice and in particular to rice plant material capable of reducing methane emissions from rice paddies.

The present invention is based on the discovery that the interaction between rice plants and methanogens is based on organic acids and in particular fumarate and other organic acids of the Krebs cycle, such as malate, secreted from the roots of the rice plants. The secreted organic acids, such as fumarate and/or malate, is in turn used as substrate by the methanogens for methanogen propagation. Hence, by reducing secretion of, in particular, fumarate from the roots of the rice plants, the amount of methanogens present in the soil in connection with the rice roots is significantly reduced as is the methane emission from the methanogens. As a consequence, a reduction in fumarate secretion from the rice roots is an effective means of achieving a low-methane rice that leads to a reduction in methane emissions from rice paddies.

Hence, an aspect of the invention relates to a method of producing a low-methane rice plant. The method comprises modifying a rice plant material for reduced organic acid secretion from roots of the rice plant material or from roots of a rice plant obtained from the rice plant material. In this embodiment, an amount of organic acids secreted from the roots of the rice plant material or of the rice plant is equal to or less than 90% of an amount of the organic acids secreted from roots of a corresponding wild-type rice plant lacking the modification. The reduced organic acid secretion from the roots of the rice plant material or of the rice plant induces a reduction in methane emission from methanogens present in connection with the roots of the rice plant material or of the rice plant.

In an embodiment, modifying the rice plant material comprises modifying the rice plant material for reduced secretion of fumarate and/or malate, preferably fumarate, from the roots of the rice plant material or from the roots of the rice plant.

Methanogens are microorganisms that produce methane as a metabolic byproduct in hypoxic conditions. They are prokaryotic and belong to the domain of archaea. Common methanogens associated with rice belong to the families Methanosaetaceae, Methanosarcinaceae, Methanobacteriales and Methanomicrobiales.

The reduction in organic acids secretion, such as fumarate secretion and/or malate secretion, by the roots of rice plants can be achieved according to various embodiments.

In an embodiment, an enzyme involved in the Krebs cycle, also referred to as the citric acid cycle (CAC) or the tricarboxylic acid cycle (TCA), is downregulated. In a particular embodiment, the enzyme is selected from the group consisting of citrate synthase, isocitrate dehydrogenase, α-ketoglutarate dehydrogenase, succinic dehydrogenase, fumarase and malate dehydrogenase.

Hence, in an embodiment, modifying the rice plant material comprises downregulating an enzyme involved in the Krebs cycle in the rice plant material. In a particular embodiment, the enzyme is preferably selected from the group consisting of citrate synthase, isocitrate dehydrogenase, α-ketoglutarate dehydrogenase, succinic dehydrogenase, fumarase and malate dehydrogenase.

Citrate synthase (E.C. 2.3.3.1) catalyzes the condensation reaction of the two-carbon acetate residue from acetyl coenzyme A (acetyal-CoA) and a molecule of four-carbon oxaloacetate to form the six-carbon citrate: acetyl-CoA+oxaloacetate+H₂O→citrate+CoA-SH.

Isocitrate dehydrogenase (IDH) (EC 1.1.1.42 and EC 1.1.1.41) is an enzyme that catalyzes the oxidative decarboxylation of isocitrate, producing alpha-ketoglutarate (α-ketoglutarate) and CO₂.

α-ketoglutarate dehydrogenase (EC 1.2.4.2, EC 2.3.1.61 and EC 1.8.1.4), also referred to as the oxoglutarate dehydrogenase complex (OGDC), is an enzyme complex catalyzing the reaction: α-ketoglutarate+NAD⁺+CoA→Succinyl-CoA+CO₂+NADH. The complex is composed of three components: oxoglutarate decarboxylase (OGDH) (EC 1.2.4.2), dihydrolipoyl succinyltransferase (DLST) (EC 2.3.1.61) and dihydrolipoyl dehydrogenase (DLD) (EC 1.8.1.4).

Succinic dehydrogenase (EC 1.3.5.1), also referred to as succinate dehydrogenase (SDH), succinate-coenzyme Q reductase (SQR) or respiratory Complex II, catalyzes the oxidation of succinate to fumarate with the reduction of ubiquinone to ubiquinol.

Fumarase (EC 4.2.1.2), also referred to as fumarate hydratase, is an enzyme that catalyzes the reversible hydration/dehydration of fumarate to malate.

Malate dehydrogenase (MDH) (EC 1.1.1.37) is an enzyme that reversibly catalyzes the oxidation of malate to oxaloacetate using the reduction of NAD⁺ to NADH.

In another particular embodiment, the enzyme is citrate synthase.

In another embodiment, multiple, i.e., at least two, enzymes involved in the Krebs cycle are downregulated, such as at least two of the above mentioned enzymes.

The downregulation of the enzyme can be done at the transcription level, at the translation level, at the post-processing level and/or at the enzyme or protein level.

A downregulation at the transcription level means that the transcription of the gene encoding the enzyme is downregulated relative to the transcription level seen in a wild-type rice material without any downregulation. The downregulation may, for instance, be achieved by replacing the wild-type or native promoter of the gene encoding the enzyme with a weaker promoter having less activity in the rice material as compared to the wild-type promoter or by an inducible promoter. Non-limiting, but illustrative, examples of such promoters include plant transcription promoters, such as the SUSIBA2 promoter and the WRI1 promoter. An alternative, or additional, way of performing enzyme downregulation at the transcription level is to remove at least a portion of any enhancer element associated with the wild-type promoter of the gene encoding the enzyme, thereby reducing the activity of the wild-type promoter and the transcription of the gene encoding the enzyme.

Hence, in an embodiment, downregulating the enzyme comprises replacing a promoter of a gene encoding the enzyme with a promoter having less activity in the rice plant material as compared to the promoter of the gene.

A downregulation at the translation level can be achieved by inhibiting or interfering with the translation of the mRNA molecules obtained following transcription of the gene encoding the enzyme. A typical example of such a translation inhibition or interference is achieved by using RNA interference (RNAi). RNAi is based on using RNA molecules inhibiting translation by neutralizing targeted mRNA molecules. Such RNAi can be achieved using RNA molecules, such as micro RNA, complementary to and thereby capable of base pairing with the mRNA molecules obtained in the transcription of the gene encoding the enzyme.

Hence, in an embodiment, downregulating the enzyme comprises interfering with translation of messenger ribonucleic acid (mRNA) molecules obtained following transcription of a gene encoding the enzyme using RNAi and an RNA molecule complementary to and capable of base pairing the mRNA molecules.

A downregulation at the post-processing level means any interference in the processes from translation of the mRNA molecules obtained following transcription of the gene encoding the enzyme up to obtaining a fully functional enzyme present in a correct position in the rice cell to catalyze its associated chemical reaction as described in the foregoing. Such downregulation may, for instance, involve interference in the transport of the enzyme from the cytosol, where translation takes place, to an organelle, such as a mitochondrion, in which the enzyme catalyzes its associated chemical reaction. Another example of downregulation is any inhibition of post-translational processing of the amino acid sequence to obtain the functional enzyme.

A downregulation at the protein or enzyme level could be achieved by additional of an inhibitor of the enzyme that is capable of binding to the enzyme and thereby competing with the target molecule of the enzyme, i.e., preventing or at least inhibiting binding of the target molecule to the enzyme. Another example of an enzyme inhibitor is an inhibitor capable, upon binding to the enzyme molecule, of inducing a change in the conformational state of the enzyme molecule and where such a change causes a reduction in the enzymatic activity of the enzyme.

Another way of reducing organic acids secretion from rice plants is to screen for rice plants with low organic acid secretion, such as fumarate secretion and/or malate secretion, from the root of the plants. This screening can be done among available rice plants. In another embodiment, a rice mutant population having low organic acids secretion can be created by, for instance, ethyl methanesulfonate (EMS) induced mutagenesis. In such a case, seeds of rice plants can be treated with EMS and then planted and selected to establish stable rice populations. The stable rice populations can then be monitored for organic acids secretion, such as fumarate secretion and/or malate secretion, from the roots and the rice populations showing a reduction in organic acids secretion from the roots over wild-type rice can be selected and optionally crossbred.

A further way of reducing organic acids secretion, such as fumarate secretion and/or malate secretion, from the roots of rice plants is to inhibit the secretion of the organic acids, such as fumarate and/or malate, from the roots, i.e., the transport of fumarate and/or malate out from the cells in the roots of the rice plants. Such an inhibition can be done in a similar way to the enzymatic downregulation as discussed in the foregoing. For instance, one or more proteins involved in the transport of fumarate and/or malate from mitochondria into the cytosol and into vacuoles and then transported from a rice root cell into the cell surroundings could be downregulated.

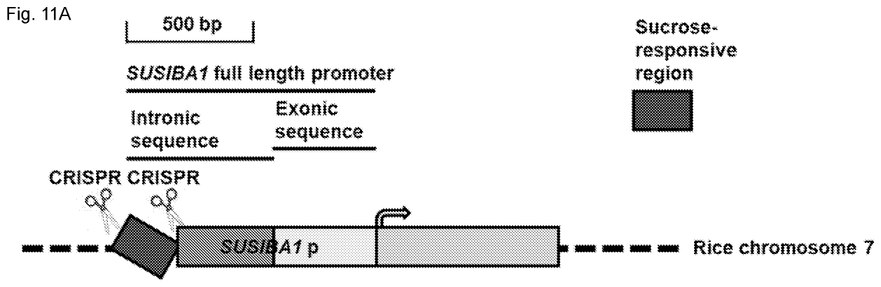

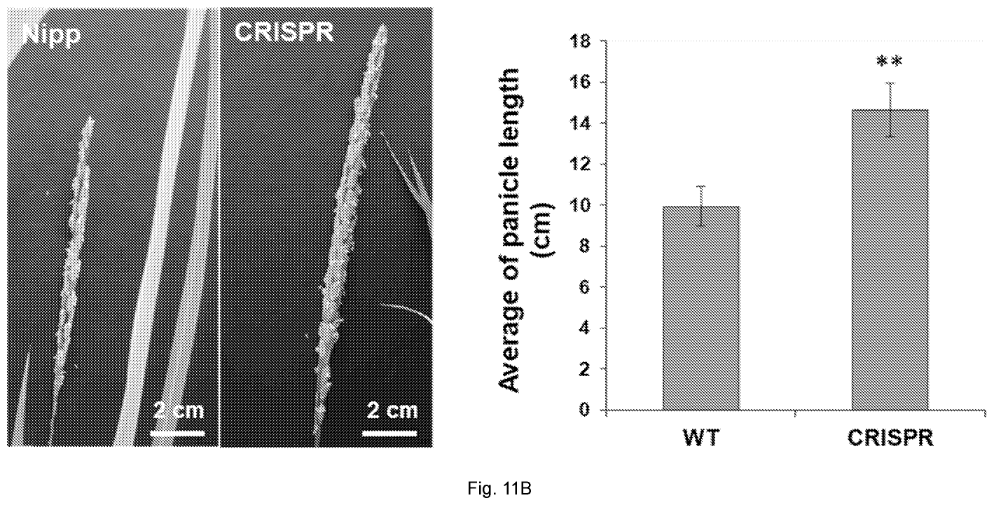

Any of the above disclosed alternatives of reducing organic acid secretion can be combined. Furthermore, any of the above disclosed alternatives of reducing organic acid secretion, including any combination thereof, can also be combined with a deletion of the yin transcription factor, sucrose-response region, or a portion thereof in the rice SUSIBA1 promoter as shown in FIG. 11A and further disclosed herein. Such a deletion of the yin transcription factor causes a carbon reallocation from the roots into the panicles. As a consequence, there is less carbon available in the roots to be used to produce organic acids and in particular fumarate, thereby resulting in a reduction in fumarate secretion.

In an embodiment, modifying the rice plant material comprises deleting a sucrose-responsive region, or a portion thereof, in the SUSIBA1 promoter in a rice plant material.

The rice SUSIBA1 promoter is present in an intron of a wild-type version of the genomic nucleotide sequence encoding the SUSIBA2 transcription factor. The absence of at least a portion of the sucrose-response region implies that any trans activation factor or complex cannot efficiently bind to the sucrose-response region and thereby cannot efficiently activate the SUSIBA1 promoter. As a consequence, no or only low amount of the SUSIBA1 transcription factor will be produced in the rice plant material regardless of the sugar level in the rice plant material. The absence or low amount of SUSIBA1 transcription factor in the rice plant material in turn implies that the SUSIBA2 transcription factor will outcompete the SUSIBA1 transcription factor for the binding to the SUSIBA2 promoter, and in more detail to the at least one W-box in the SUSIBA2 promoter. This will in turn cause activation of the SUSIBA2 promoter and further production of the SUSIBA2 transcription factor in the rice plant material. The high levels of the SUSIBA2 transcription factor and the low levels of the SUSIBA1 transcription factor in the rice plant material induces causes the above mentioned carbon reallocation in the rice plant material. For more information of the rice SUSIBA1 and SUSIBA2 genes, promoters and their transcription factor, reference is made to WO 2018/182493, the teaching of which is hereby incorporated by reference.

Another aspect of the invention relates to a method of reducing emission of methane from a rice paddy. The method comprises deleting a sucrose-responsive region in the SUSIBA1 promoter in a rice plant material. The method also comprises cultivating the rice plant material or a rice plant obtained from the rice plant material in a rice paddy.

A further aspect of the invention relates to a method of reducing emission of methane from a rice paddy. The method comprises reallocating carbon from roots of a rice plant into panicles of the rice plant. The method also comprises cultivating the rice plant in a rice paddy. In this embodiment, carbon reallocation causes a reduction in production and secretion of organic acids from the roots and thereby reduced methane emission from methanogens present in the soil in connection with the roots in the rice paddy.

A “rice plant material” is in an embodiment a rice plant. In another embodiment, a rice plant material is a rice cell, including multiple such rice cells. A rice plant material is, in a further embodiment, a rice plant tissue or organ, including but not limited to, epidermis; ground tissue; vascular tissue, such as xylem or phloem; meristematic tissues, such as apical meristem, lateral meristem or intercalary meristem; permanent tissues, such as simple permanent tissue, including for instance parenchyma, collenchyma, sclerenchyma or epidermis, complex permanent tissue, including for instance xylem, phloem, or special or secretory tissues. A rice plant material is, in yet another embodiment, a rice seed.

In an embodiment, the rice plant material is not a plant material of wild rice. Hence, the rice plant material is preferably a plant material of cultivated rice. In an embodiment, the rice plant material is an Oryza sativa plant material or an Oryza glaberrima plant material.

A “reduction of organic acids secretion”, such as a reduction of fumarate secretion, as used herein indicates a significant reduction of secretion of the organic acids, such as fumarate, from the roots of a rice plant according to the embodiments as compared to a corresponding control or wild-type rice plant. In various embodiments, the amount of organic acids, such as fumarate, secreted from the roots of a rice plant according to the embodiments could be equal to or less than 90%, preferably equal to or less than 85%, equal to or less than 80%, equal to or less than 75%, equal to or less than 70%, equal to or less than 65%, equal to or less than 60%, equal to or less than 55%, equal to or less than 50%, equal to or less than 45%, equal to or less than 40%, equal to or less than 35%, equal to or less than 30%, equal to or less than 25%, equal to or less than 20%, equal to or less than 15%, equal to or less than 10%, or even equal to or less than 5% of the amount of organic acids, such as fumarate, secreted from the roots of the control or wild-type rice plant.

In a particular embodiment, the amount of fumarate secreted from the roots a rice plant according to the embodiments is 2.5 to 5.0 fold less than the amount of fumarate secreted from the roots of the rice variety Nipponbare.

### EXAMPLES

### Example 1

SUSIBA2 rice (Su et al. (2015)) is a low-methane rice that produces 50% more filled-grain numbers with elevated starch content from 77% to 86% in the grains. Importantly, SUSIBA2 rice remarkably reduces methane emissions from rice paddies, associated with a significantly decreased growth of methanogens. However, the mechanism behind the SUSIBA2 rice methane reduction is unknown. In these Examples, ribonucleic acid (RNA) sequencing (RNAseq), microbe deoxyribonucleic acid (DNA) sequencing (DNAseq), nuclear magnetic resonance (NMR), quantitative polymerase chain reaction (qPCR), and gas chromatography (GC) were used to monitor the interactions between SUSIBA2 rice and methanogens. The results showed that interactions between SUSIBA2 rice and methanogens were organic acids mainly fumarate secreted by SUIBA2 rice. During rice cultivation, SUSIBA2 rice started to reduce methane emissions from around week 6 by reducing root growth thereby providing less physical area of habitat for methanogens and by secreting less fumarate, which can be converted to substrates for methanogen growth.

### Material and Methods

Plant Materials and Growth Conditions Rice plants of variety Nipponbare (Oryza sativa L. ssp. Japonica), abbreviated as Nipp herein, and SUSIBA2 rice were cultivated according to Su et al. (2015).

### RNAseq

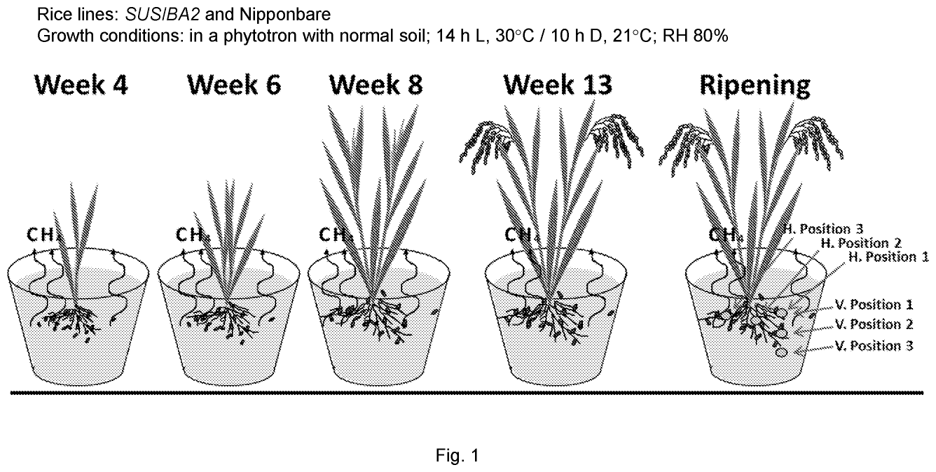

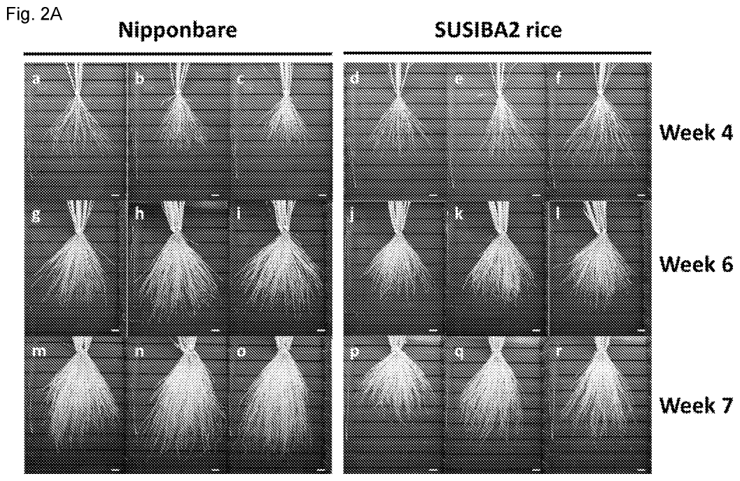

Total RNA isolation was according to Su et al. (2015). Root samples for RNA isolation were from horizontal position 1 (H. Position 1) or vertical position 1 (V. Position 1) at Week 6 as indicated in FIG. 1. RNAseq and bioinformatics were done at SciLifeLab, BMC, Uppsala University.

### Microbe DNAseq

Microbe DNA isolation from both phytotron rice soil and paddy soil were according to Su et al. (2015). DNAseq was performed at BMC, Uppsala University.

### NMR

Samples for NMR analysis were from vertical positions 1-3 at different time points during rice cultivation (FIG. 1). Sample preparation and NMR analysis were according to Coulomb et al. (2015) and Rohnisch et al. (2018).

qPCR

Quantitative PCR was performed for both methanogen determination and rice gene expression in the same way as described in Su et al. (2015).

### GC Analysis

Gas chromatography analysis to determine methane concentrations was according to Su et al. (2015).

### Results

SUSIBA2 rice and the control rice Nipponbare (Nipp) were cultivated in phytotron conditions and followed during cultivation at Week 4, Week 6, Week 8, Week 13 and Ripening stage (FIG. 1). Various experiments were conducted on samples from different spatial sites of rice rhizosphere regions, i.e., horizontal position 1-3 and vertical position 1-3, to analyze the interactions between SUSIBA2 rice and methanogens.

### Root Phenotyping

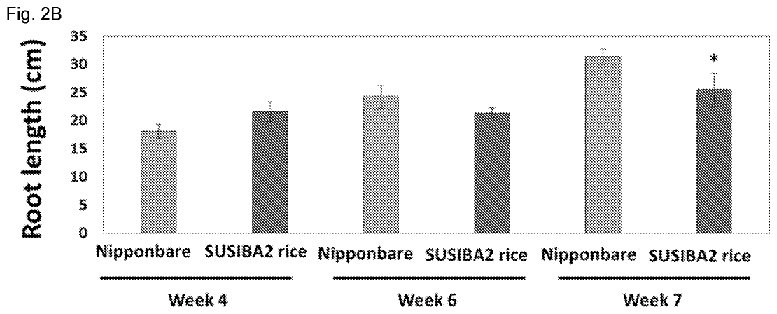

The rice root morphological changes were followed during rice cultivation. The root size of SUSIBA2 rice was the same or slightly larger than the wild-type control Nipponbare at Week 4 after planting (FIG. 2). Interestingly, after Week 6 the roots of SUSIBA2 rice grew more slowly than Nipponbare and became significantly smaller than Nipponbare at Week 7 (FIG. 2B). After Week 7 it was difficult to follow the root size development due to the large root size, but the SUSIBA2 rice had smaller root size than Nipponbare.

### Methane Emissions

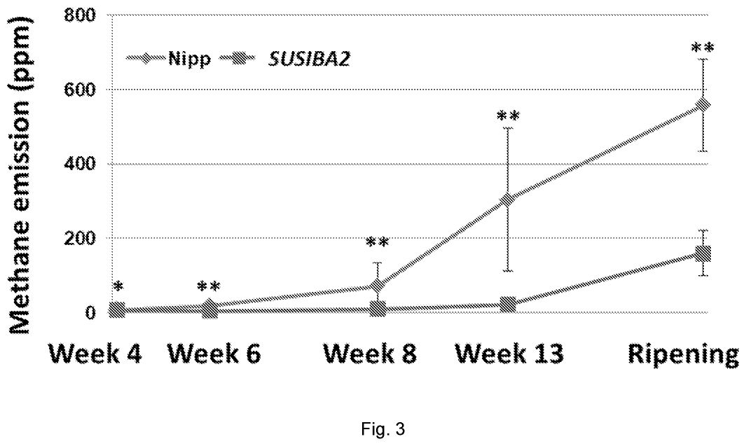

Methane emissions were measured during rice cultivation. After Week 6, the Methane Emissions were significantly reduced from SUSIBA2 rice compared with the wild type control rice Nipponbare (FIG. 3).

### Methanogen Determination

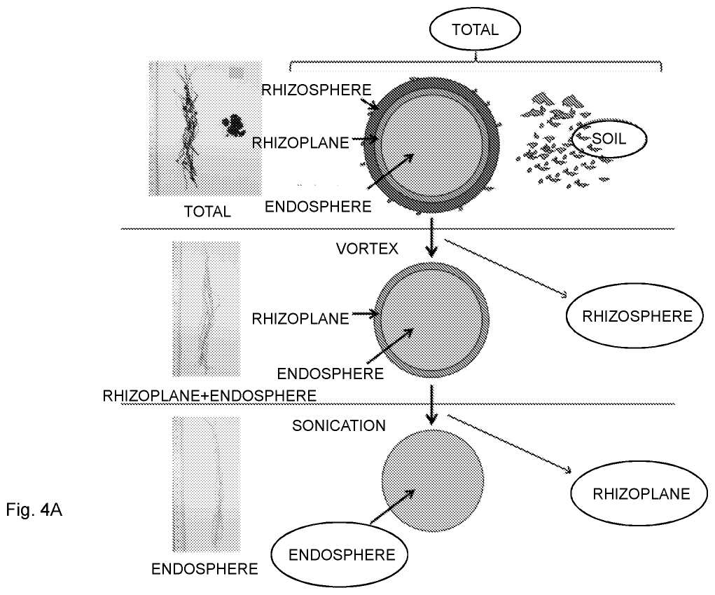

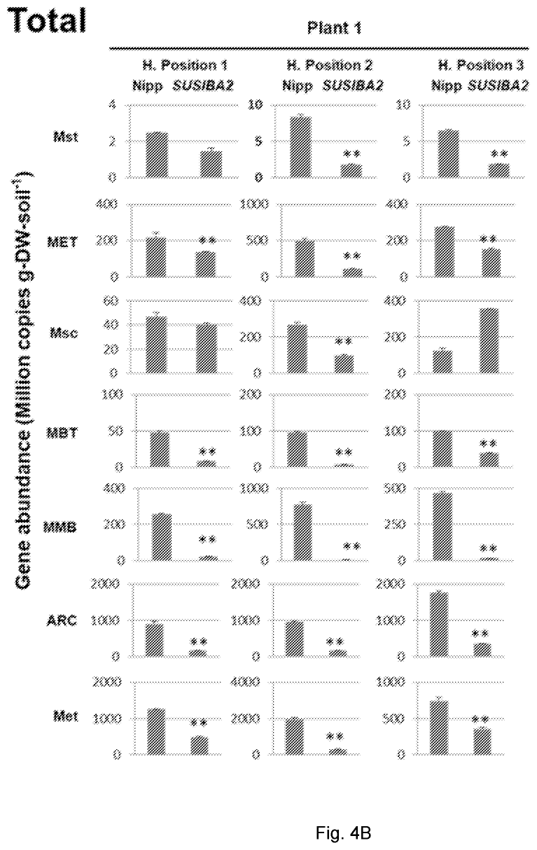

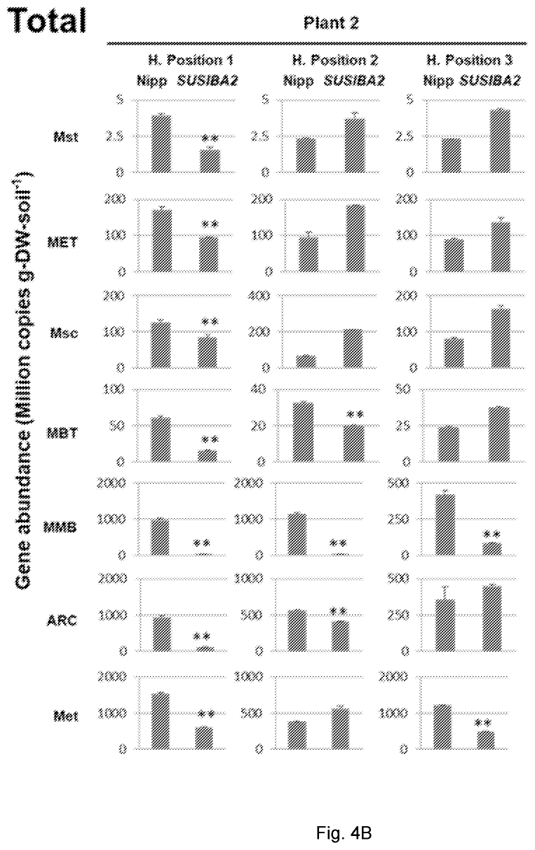

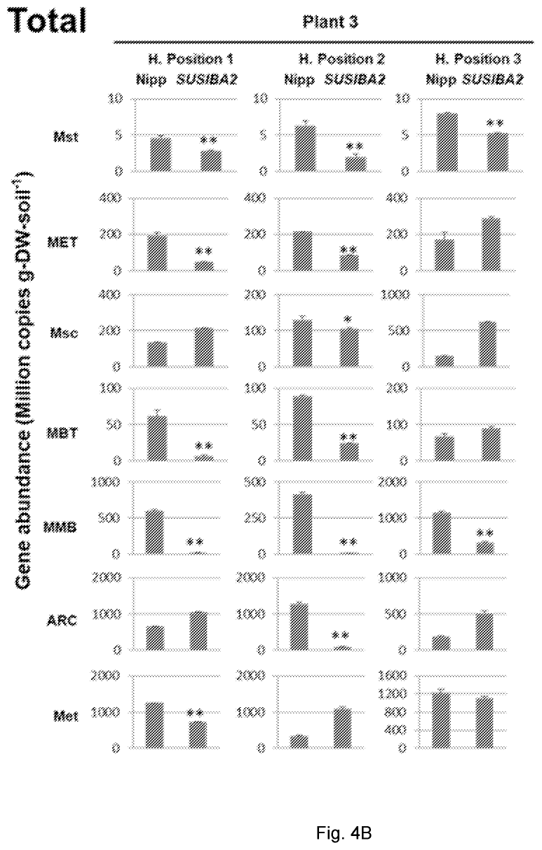

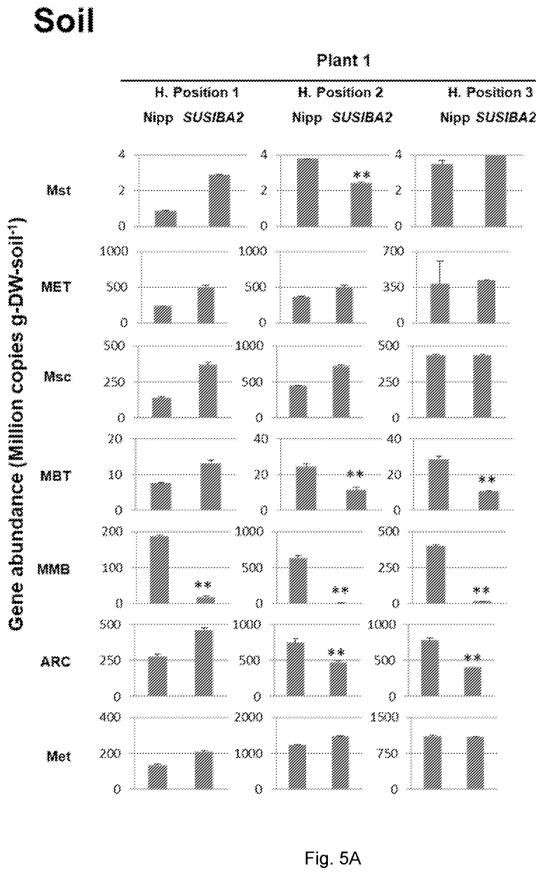

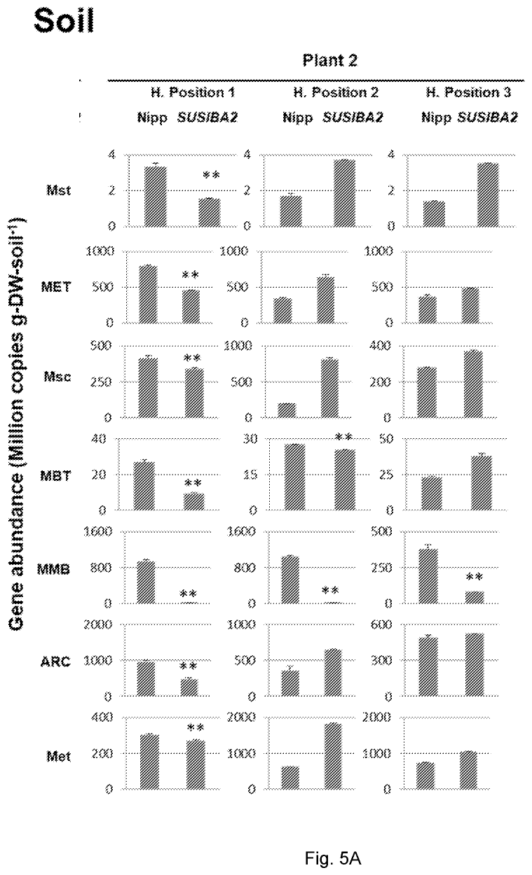

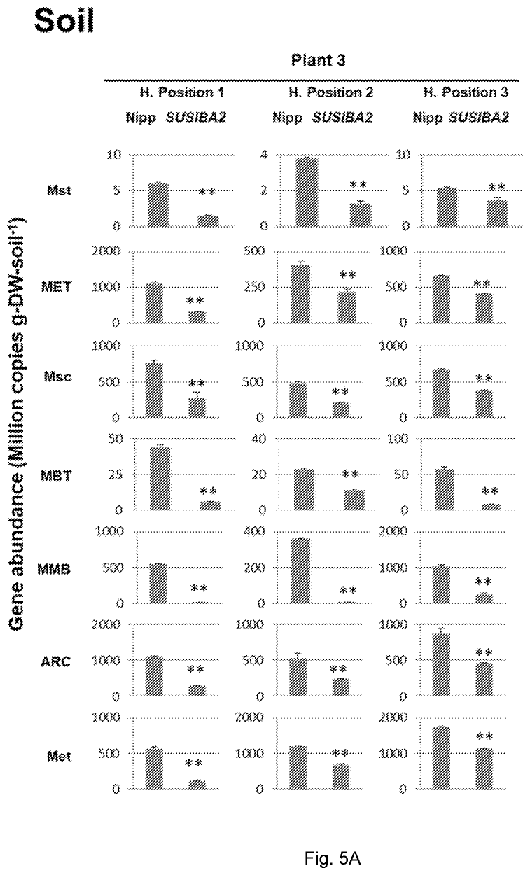

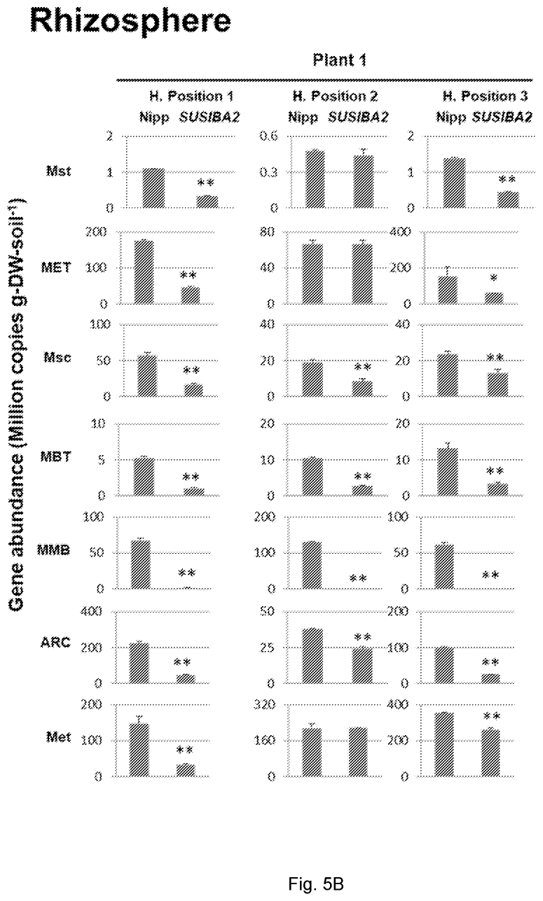

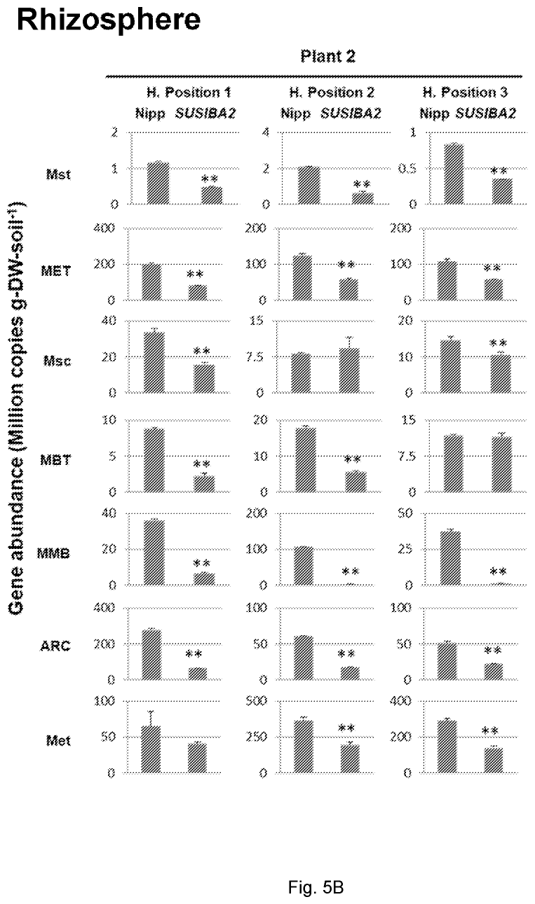

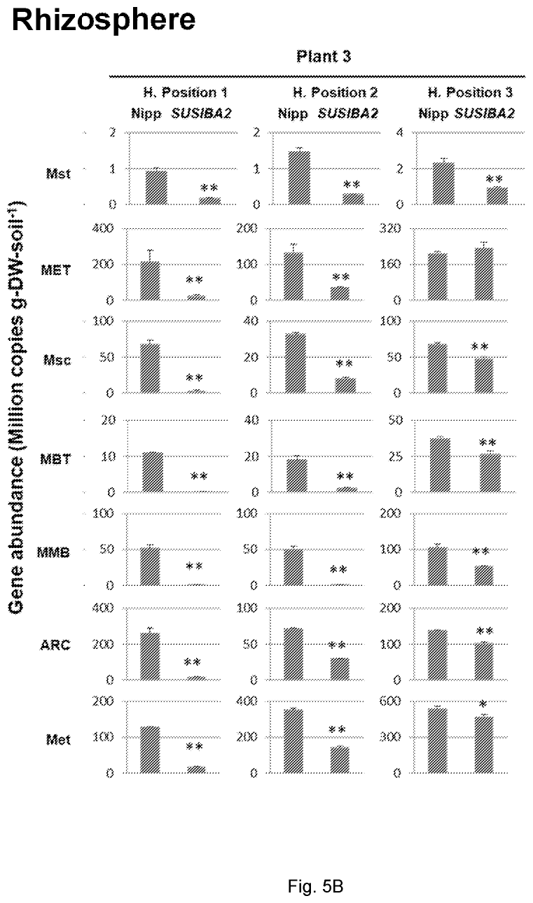

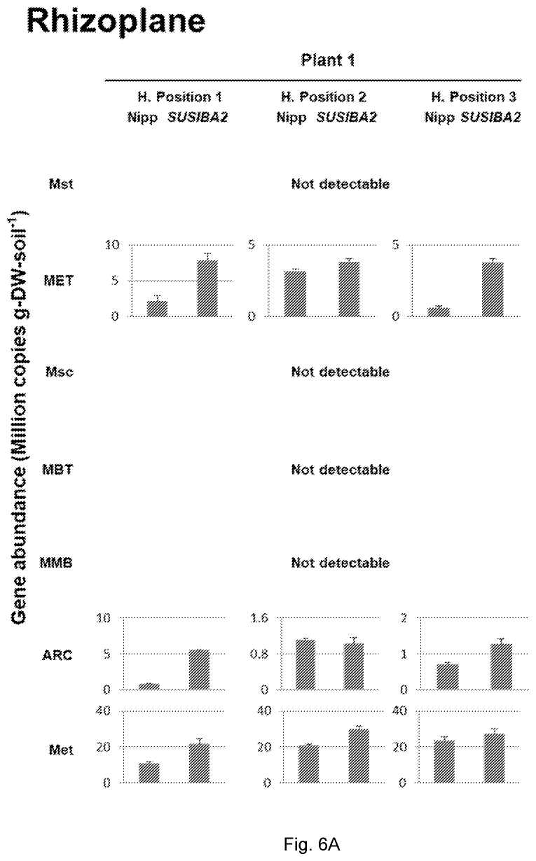

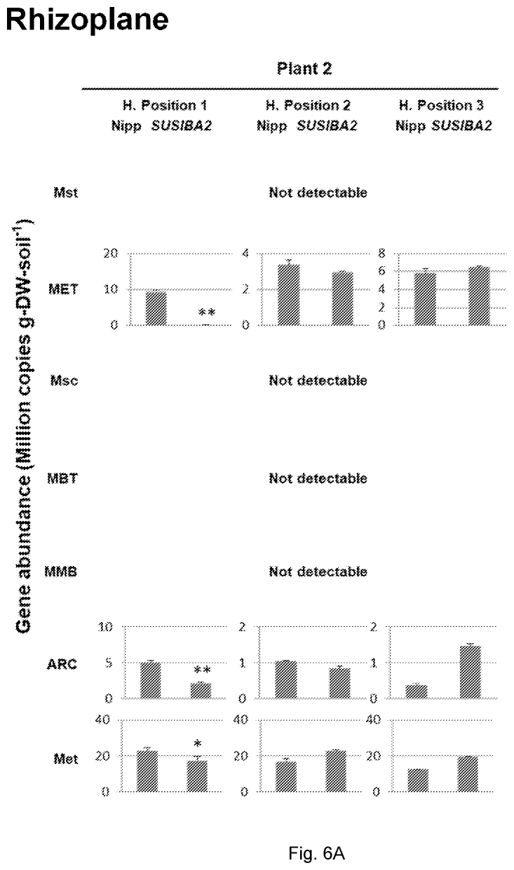

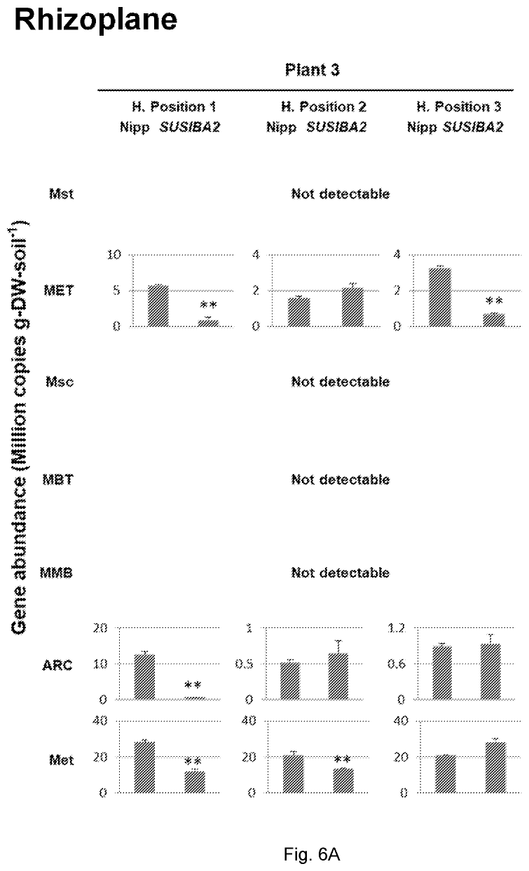

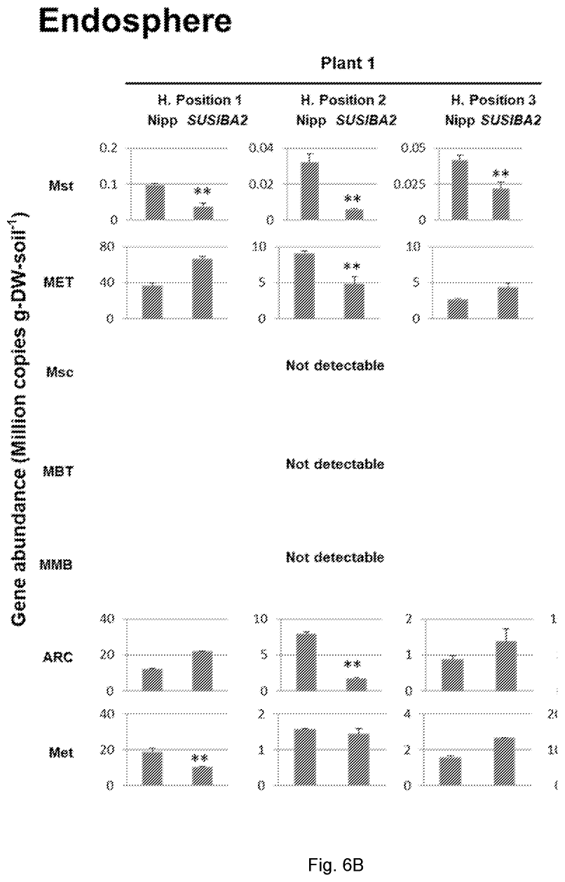

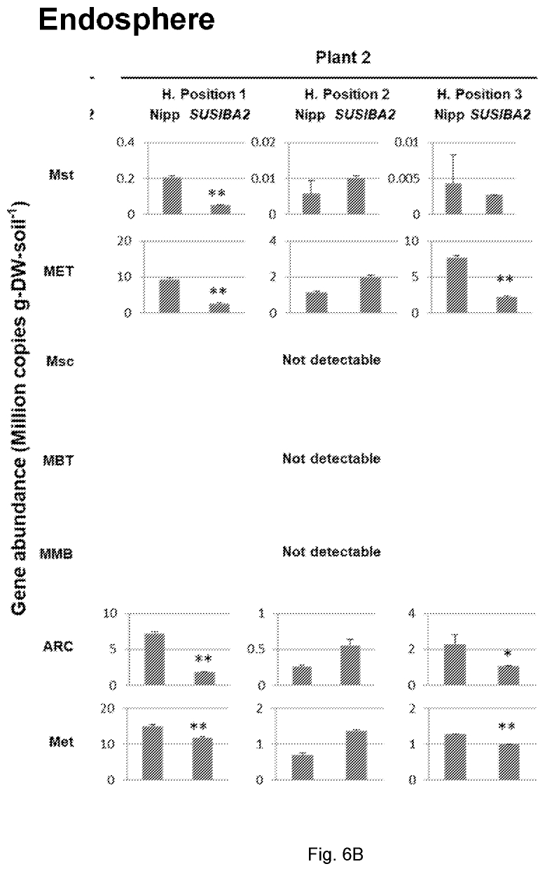

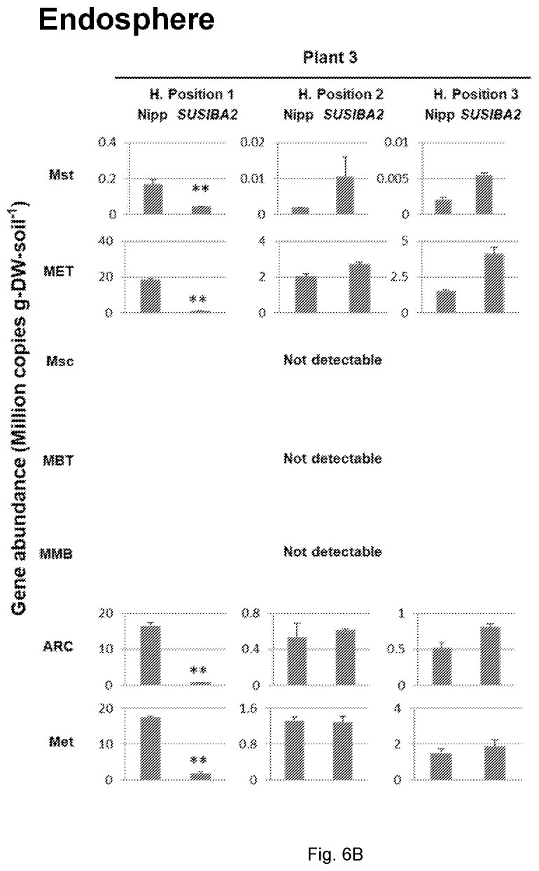

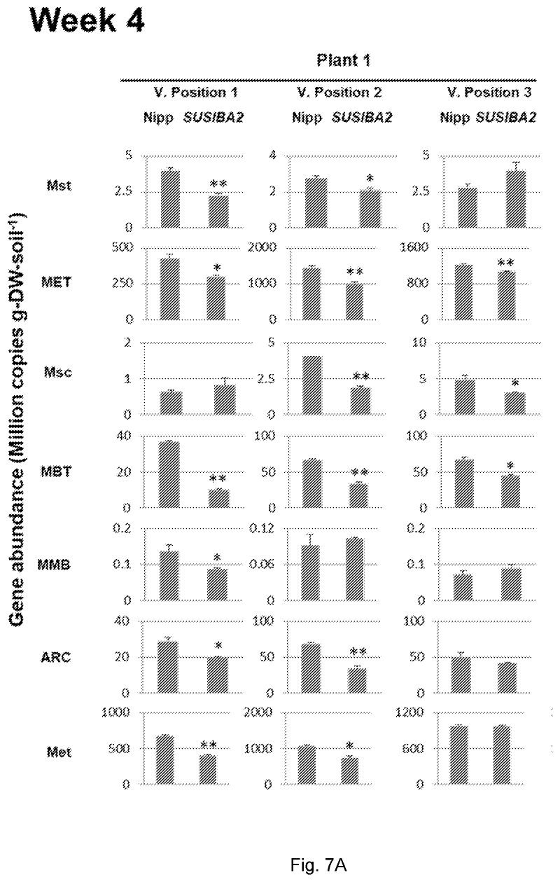

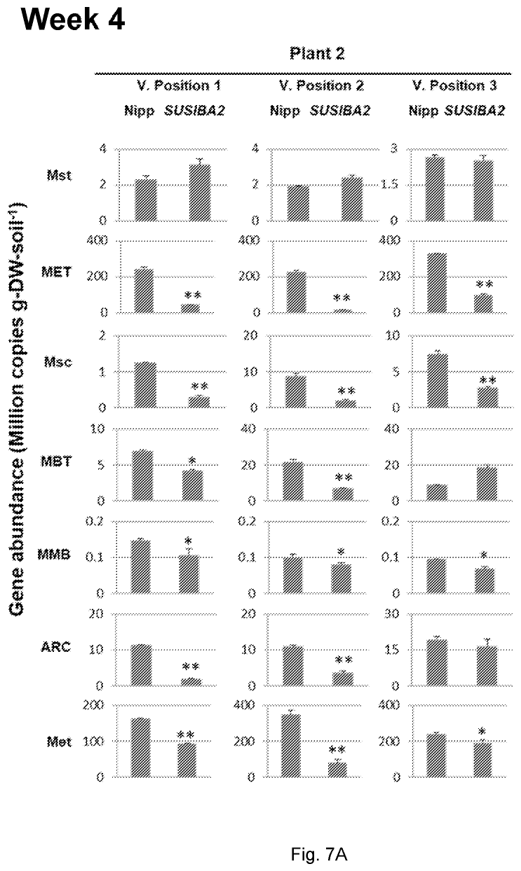

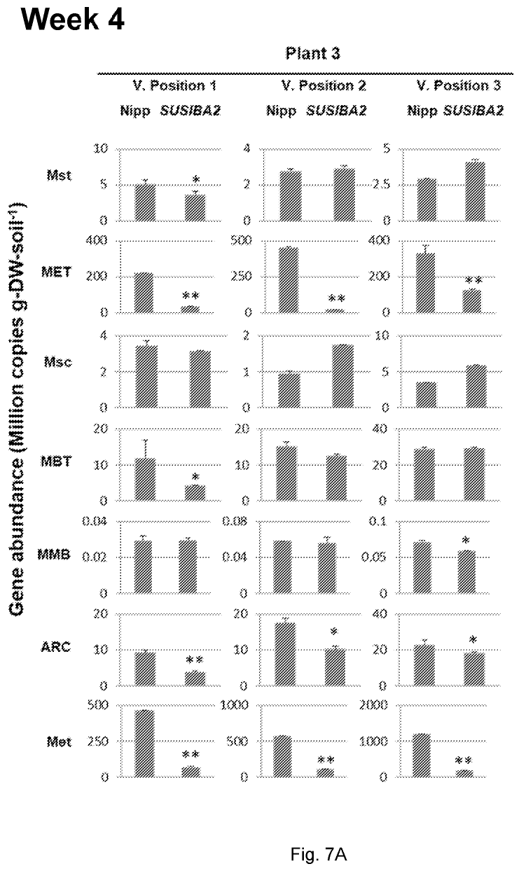

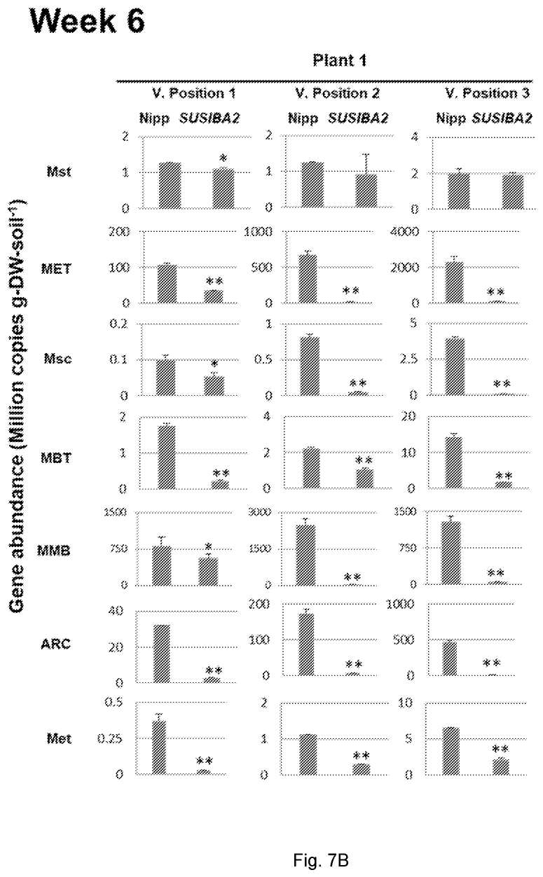

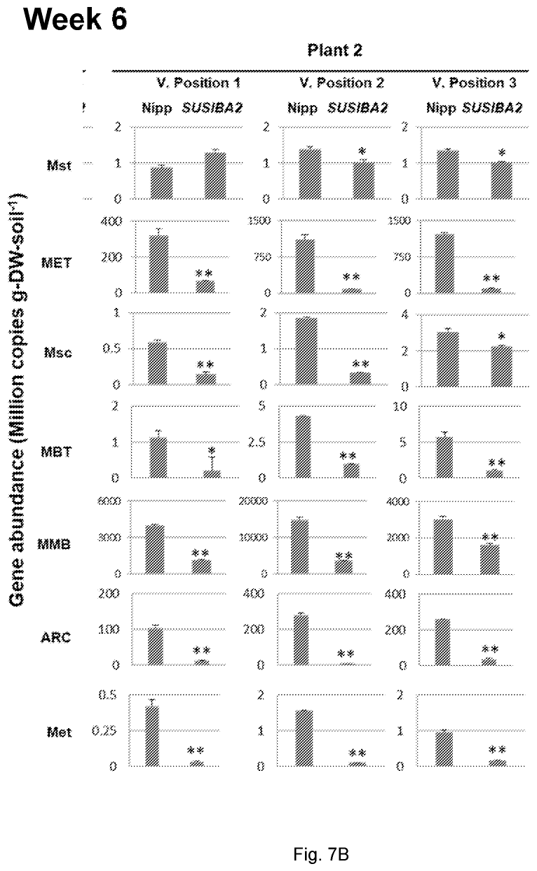

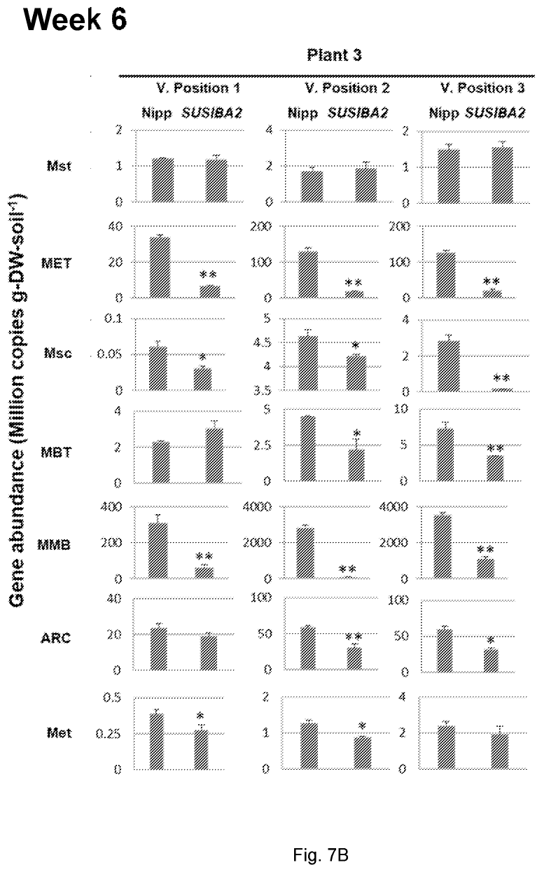

According to the protocol of Edwards et al. (2015), the rice roots and rhizosphere region soil were divided into four fractions: soil, rhizosphere, rhizoplane, and endosphere (FIG. 4A). The methanogen determination showed that there were less methanogens in the seven groups in SUSIBA2 rice than in Nipponbare in all four fractions (FIGS. 4B, 5 and 6). Interestingly, groups of microbes other than MET and Met, which are the most important or dominant methanogens in rice paddies for methane emissions, could not be detected in the fractions of rhizoplane and endosphere. Examination of the vertical positions during rice cultivation showed that there were less methanogens in the seven groups in SUSIBA2 rice than in Nipponbare in all depths from Week 4 from planting (FIG. 7).

### NMR Analysis of Root Exudates

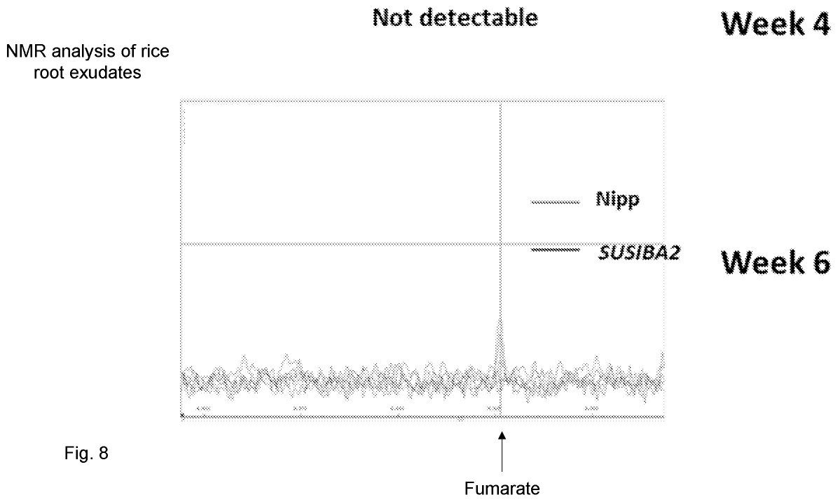

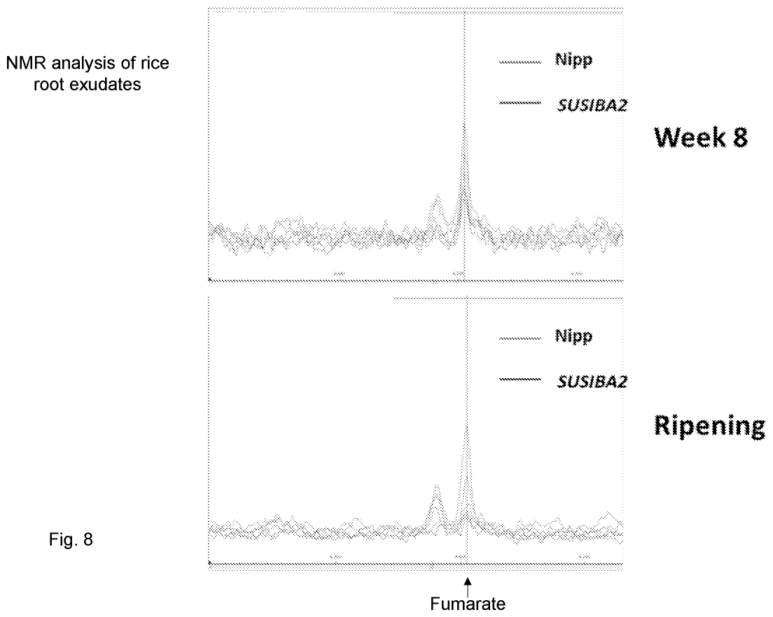

Root exudates from SUSIBA2 rice and Nipponbare were analyzed by NMR analysis. There was a significant difference in the amount of fumarate between the rice root exudates (FIG. 8). Nippponbare rice secreted more fumarate than SUSIBA2 rice from Week 6. The secreted fumarate can be converted to substrates for methanogen propagation around rhizosphere.

### RNAseq Analysis of Rice Root Transcriptome

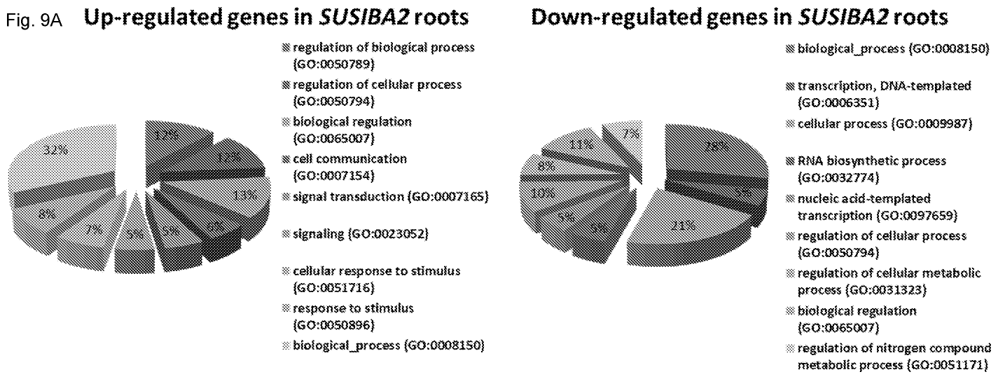

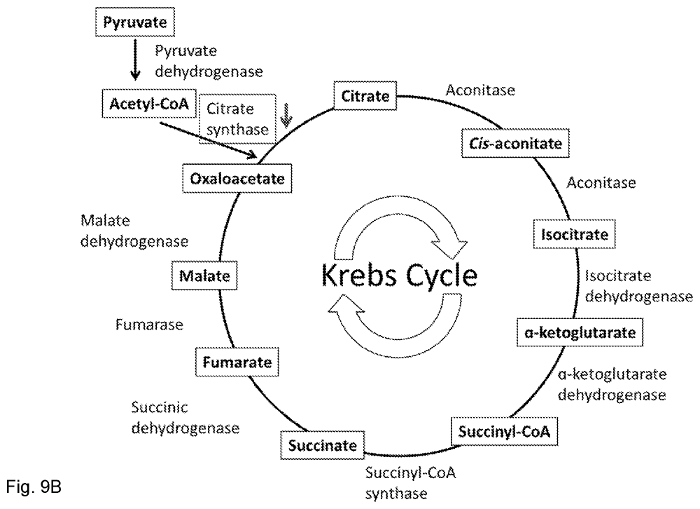

RNAseq analysis was used to detect 600 genes that were significantly (P<0.05) upregulated and 889 genes that were downregulated in SUSIBA2 rice roots (FIG. 9A). Among the downregulated genes, all fumarate synthesis related genes were examined. One of the significantly downregulated genes was the gene coding for citrate synthase (E.C. 2.3.3.1), a rate-limiting enzyme for the Krebs cycle (FIG. 9B).

### DNAseq of Microbes Around Rice Rhizosphere

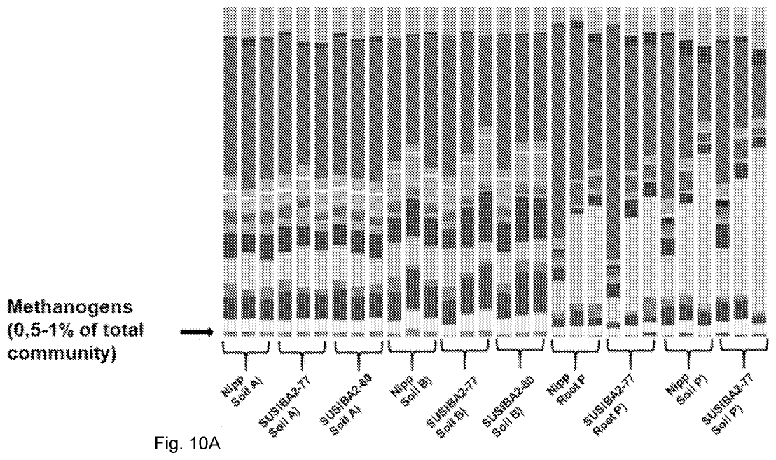

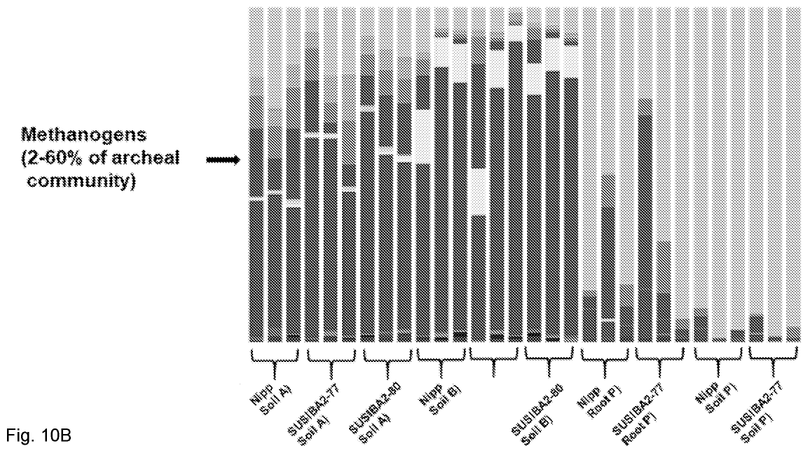

DNAseq results showed low impact on the overall community composition between the different rice varieties, illustrating that SUSIBA2 rice did not cause any major changes in the soil microbe community. The major difference was actually shown between phytotron samples and field samples. The results also showed that the total relative abundance of methanogens in relation to the bacterial community was very low, below 1%, which is a normal situation for this type of community. Still methanogens comprised up to 60% of the archaeal community. Also, in line with the qPCR analyses, the relative abundance of the methanogens was significantly lower for the SUSIBA2 rice as compared with Nipponbare rice (FIG. 10).

### Conclusions

The experiments showed that the interactions between SUSIBA2 rice and methanogens are organic acids and mainly fumarate. The reduction in methane emissions as seen in SUSIBA2 rice is caused by this reduction in secretion of fumarate around Week 6 after planting. Secreted fumarate can be converted to substrates for methanogen propagation. Hence, a reduction in fumarate secretion reduces the amount of substrates for methanogens. Furthermore, also around Week 6 after planting, SUSIBA2 rice started to reduce root size growth and thereby provided less physical habitat area for methanogen growth.

### Example 2

Carbon allocation from biomass below ground to biomass above ground can be used to reduce fumarate secretion from roots below ground, which limits the growth of the methanogenic community in the rice rhizosphere.

### Material and Methods

Clustered regularly interspaced short palindromic repeats (CRISPR)/CRISPR-associated protein (Cas) deletion was done at Biogle Genome Editing Center, China, to delete the yin transcription factor (sucrose-responsive region) in the rice SUSIBA1 promoter of Nipponbare rice (Lu et al. (2017); Jin et al. (2017)). qPCR analysis of relative gene expression was performed according to Jin et al. (2017).

### Results

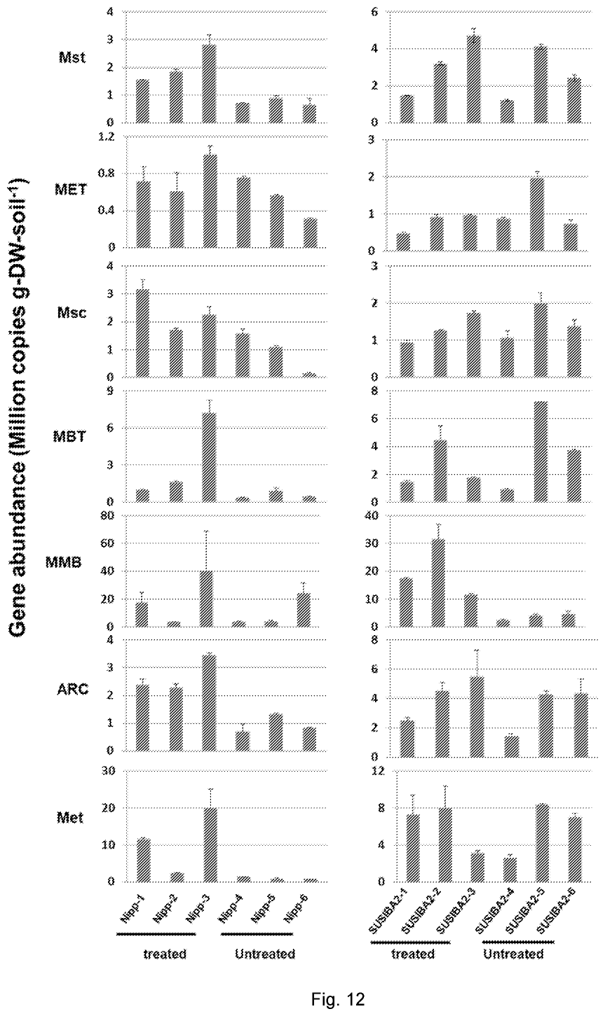

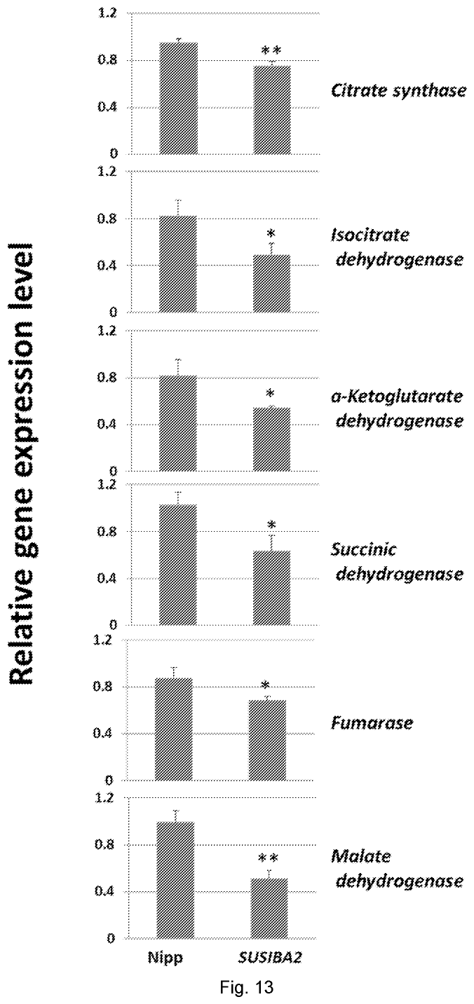

CRISPR/Cas technology was used to successfully delete the yin transcription factor (sucrose-response region) in the yin-yang system (Jin et al. (2017)) in rice to achieve larger panicles (FIG. 11). The increased panicle size meant that more carbon was reallocated to the panicles, which in turn led to less carbon to fumarate section from roots below ground. When SUSIBA2 rice and Nipponbare rice were treated with fumarate, all methanogen groups propagated (FIG. 12), indicating that fumarate could be converted to substrates for methanogen growth.

In the RNAseq experiments (FIG. 9), only the gene coding for citrate synthase was found to be downregulated and genes for other enzymes in the Krebs cycle were not found due to lack of annotation in the Nipponbare genome sequence database. When qPCR was used to analyze genes for two other rate-limiting enzymes, isocitrate dehydrogenase and α-ketoglutarate dehydrogenase and genes for key enzymes in fumarate accumulation, all the analyzed genes were downregulated in SUSIBA2 rice roots, indicating that these genes can be used as probes for screening for low root fumarate rice.

### Example 3

Geobacter species are environmentally important, in part because of their ability to anaerobically oxidize acetate with the reduction of extracellular electron acceptors, such as Fe(III) and Mn(IV) oxides, humic substances, U(VI), and graphite electrodes. Some Geobacter species, including Geobacter sulfurreducens, are also able to use the tricarboxylic acid (TCA) cycle intermediate fumarate as an electron acceptor and acetate as a donor. It has been demonstrated that G. sulfurreducens has only one enzyme, FrdCAB, that functions in vivo as fumarate reductase.

### Materials and Methods

Samples from the rhizosphere part of root at proximal regions 5 cm from up phase (V. Position 1 in FIG. 1) of four independent plants for Nipponbare (Nipp) and SUSIBA2 were collected at 3.00 pm from phytotrons and then the samples were used to isolate soil DNA using a DNA isolation kit for soil organisms (FastDNA SPIN Kit for Soil; MP Biomedicals, LLC). The DNA was then quantified and adjusted into the same concentrations. qPCR quantification was performed using a standard of a cloned 16S rRNA gene fragment of Geobacter sulfurreducens with the primers G.Sulf923F: TGACATCCACGGAACCCTCC (SEQ ID NO: 1); G.Sulf1399R: GACGCTGCCTCCATTGCTG (SEQ ID NO: 2). The abundance of Geobacter sulfurreducens was calculated and translated to DNA copy numbers for each gram of dry soil sample. The qPCR program for Geobacter sulfurreducens was as follows: 95° C. for 7 min, then 40 cycles of 10 s at 95° C., 40 s at 60° C. and 40 s at 72° C. All melting curves are from 55° C. to 95° C. with an increase of 0.05° C. per second.

### Results

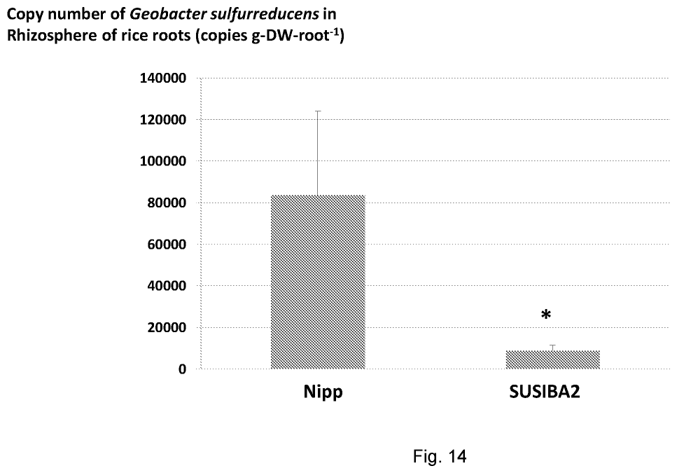

qPCR analysis showed that the group of Geobacter, Geobacter sulfurreducens, associated with the fraction of rhizosphere (see FIG. 4A) in Nipponbare roots was significantly more than in SUSIBA2 rice (FIG. 14). This means the Nipponbare rice secreted more fumarate than the SUSIBA2 rice, which led to more Geobacter sulfurreducens to generate acetate for methanogens. Accordingly, compared with the SUSIBA2 rice, the control Nipponbare rice secreted more fumarate, which stimulated Geobacter sulfurreducens to produce more acetate. More acetate as methanogenic substrates enriched methanogens to release more methane.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible.

### REFERENCES

Coulomb et al. (2015) Metabolomics study of cereal grains reveals the discriminative metabolic markers associated with anatomical compartments, Italian Journal of Food Science 27:1-9
Edwards et al. (2015) Structure, variation, and assembly of the root-associated microbiomes of rice, The Proceedings of the National Academy of Sciences of the United States of America 112: E911-E920
Lu et al. (2017) Genome-wide Targeted Mutagenesis in Rice Using the CRISPR/Cas9 System, Molecular Plant 10: 1242-1245
Jin et al. (2017) A Dual-Promoter Gene Orchestrates the Sucrose-Coordinated Synthesis of Starch and Fructan in Barley, Molecular Plant 10: 1556-1570
Rohnisch et al. (2018) A QuA: An Automated Quantification Algorithm for High-Throughput NMR-Based Metabolomics and Its Application in Human Plasma, Analytical Chemistry 90: 2095-2102
Su et al. (2015) Expression of barley SUSIBA2 transcription factor yields high-starch low-methane rice, Nature 523: 602-606

1.-11. (canceled)

12. A method of producing a low-methane rice plant, the method comprising modifying a rice plant material for reduced organic acid secretion from roots of the rice plant material or from roots of a rice plant obtained from the rice plant material, wherein
an amount of organic acids secreted from the roots of the rice plant material or of the rice plant is equal to or less than 90% of an amount of the organic acids secreted from roots of a corresponding wild-type rice plant lacking the modification; and
the reduced organic acid secretion from the roots of the rice plant material or of the rice plant induces a reduction in methane emission from methanogens present in connection with the roots of the rice plant material or of the rice plant.

13. The method according to claim 12, wherein the amount of organic acids secreted from the roots of the rice plant material or of the rice plant is equal to or less than 80% of the amount of the organic acids secreted from roots of the corresponding wild-type rice plant lacking the modification.

14. The method according to claim 13, wherein the amount of organic acids secreted from the roots of the rice plant material or of the rice plant is equal to or less than 70% of the amount of the organic acids secreted from roots of the corresponding wild-type rice plant lacking the modification.

15. The method according to claim 14, wherein the amount of organic acids secreted from the roots of the rice plant material or of the rice plant is equal to or less than 60% of the amount of the organic acids secreted from roots of the corresponding wild-type rice plant lacking the modification.

16. The method according to claim 12, wherein modifying the rice plant material comprises deleting a sucrose-responsive region, or a portion thereof, in the SUSIBA1 promoter in a rice plant material.

17. The method according to claim 12, wherein modifying the rice plant material comprises downregulating an enzyme involved in the Krebs cycle in the rice plant material.

18. The method according to claim 17, wherein the enzyme is selected from the group consisting of citrate synthase, isocitrate dehydrogenase, □-ketoglutarate dehydrogenase, succinic dehydrogenase, fumarase and malate dehydrogenase.

19. The method according to claim 17, wherein downregulating the enzyme comprises replacing a promoter of a gene encoding the enzyme with a promoter having less activity in the rice plant material as compared to the promoter of the gene.

20. The method according to claim 17, wherein downregulating the enzyme comprises interfering with translation of messenger ribonucleic acid (mRNA) molecules obtained following transcription of a gene encoding the enzyme using RNA interference (RNAi) and an RNA molecule complementary to and capable of base pairing the mRNA molecules.

21. The method according to claim 12, wherein modifying the rice plant material comprises modifying the rice plant material for reduced secretion of fumarate and/or malate from the roots of the rice plant material or from the roots of the rice plant.

22. The method according to claim 21, wherein modifying the rice plant material comprises modifying the rice plant material for reduced secretion of fumarate from the roots of the rice plant material or from the roots of the rice plant.

23. The method according to claim 12, wherein the rice plant material is an Oryza sativa plant material or an Oryza glaberrima plant material.

24. The method according to claim 12, wherein the rice plant material is selected from the group consisting of a rice plant, a rice cell, a rice plant tissue or organ, and a rice seed.

25. A method of reducing emission of methane from a rice paddy, the method comprising:
deleting a sucrose-responsive region in the SUSIBA1 promoter in a rice plant material; and
cultivating the rice plant material or a rice plant obtained from the rice plant material in a rice paddy.

26. A method of reducing emission of methane from a rice paddy, the method comprising:
reallocating carbon from roots of a rice plant into panicles of the rice plant; and
cultivating the rice plant in a rice paddy, wherein carbon reallocation causes a reduction in production and secretion of organic acids from the roots and thereby reduced methane emission from methanogens present in the soil in connection with the roots in the rice paddy.
